(19) United States
(12) Patent Application Publication
(10) Pub. No.: US 2015/0088137 A1
(43) Pub. Date: Mar. 26, 2015

(54) ULTRASONIC SURGICAL INSTRUMENT WITH DUAL END EFFECTOR

(71) Applicant: MISONIX INCORPORATED, Farmingdale, NY (US)

(72) Inventor: Ronald Manna, Valley Stream, NY (US)

(73) Assignee: MISONIX INCORPORATED, Farmingdale, NY (US)

(21) Appl. No.: 14/038,394

(22) Filed: Sep. 26, 2013

(51) Int. Cl. A61B 17/16; A61B 17/32

(52) U.S. Cl. CPC A61B 17/16; A61B 17/320068; A61B 2017/1651
USPC 606/79

(57) ABSTRACT

An ultrasonic surgical instrument includes at least two tools each having a respective operating head with an operative surface or edge. At least one source of ultrasonic mechanical vibratory energy is provided which produces at least two vibration modes out of phase with one another. The tools are each connected to the source of ultrasonic mechanical vibratory energy for enabling transmission of a respective one of the vibration modes into the respective tool so that the tools are driven out of phase with one another.

### FIELD OF THE INVENTION

This invention relates to an ultrasonic surgical instrument. More particularly, this invention relates to an ultrasonic surgical instrument with dual end effectors. Where the instrument particularly takes the form of an instrument to cut tissue such as cartilage and bone, the dual end effectors are parallel cutting blades.

### BACKGROUND OF THE INVENTION

In the field of orthopedics, the cutting of living bone is a prerequisite for many procedures. Such procedures include the reconstruction of damaged tissue structures due to accidents, the grafting of healthy bone into areas damaged by disease, or the correction of congenital facial abnormalities like a receding chin line. Over several centuries, these tasks were performed through the utilization of devices called bone saws.

Traditional bone saws are categorized into several basic categories. Hand powered saws or drills are just that, hand held devices which require the operator to move the device in a fashion similar to that used for carpentry tools. Powered devices, whether electric or pneumatic, are of either the reciprocating or rotary type. The reciprocating devices use a flat, sword like blade where the back and forth motion is provided by a motor instead of the hand. The rotary devices use a rotating motor to spin a drill bit or a blade which has teeth arranged around its circumference similar to a table saw blade. All of these traditional bone saws are used today in medical procedures around the world.

While traditional saws are functional, they have many disadvantages. With either the band or reciprocating saws, for instance, it is not easy to initiate and direct a cut. A cut must start from an edge or, alternatively, a starting hole must be used. To create a starting hole, a drill or similar instrument is operated to bore into the bone. Subsequently, a cutting blade is inserted into the bored hole. The user can then proceed to cut. Alternatively, a rotary type blade may be used. However, when a rotary blade is used, the cut must follow a relatively straight path to prevent the blade from binding in the cut. With all blades the ability to create a curved or compound angle cut is extremely limited by the blade chosen. The relatively thick blades have a wide kerf; so that a significant thickness of the viable bone is lost in the cutting procedure. Physicians would like this width to be as thin as possible in most procedures where reconstruction is necessary.

Above all, the relatively slow linear or tangential speeds of conventional bone saw blades coupled with the teeth necessary for cutting result in high frictional losses, which becomes manifested as heat. Heat will cause necrosis of the tissue if the bone temperatures reach 47EC for more than a few seconds. When tissue necroses, the bone recedes after the surgery as the necrotic bone is overgrown. During such natural post-surgical tissue developments, the thickness of the cuts in the bone actually increases. The bone rescission process must be complete before healing can begin. To prevent the shortening of the length of the bone, metal plates and screws are used to fix the bone fragments in proper position. All of these factors obviously lead to increased operative time, and more importantly, to dramatically increased healing time, since the bone must knit across a greater span. Some studies have shown the strength of the bone to be effected negatively as well

To limit the tissue temperature rise in an attempt to reduce necrosis, some traditional surgical saws provide cooling liquid to the surgical site. Several researchers have proposed the use of ultrasonic tools for bone separation. The use of ultrasonic surgical instruments for cutting through various tissues is well known. While these devices are superior to the traditional saws in several aspects such as reduced kerf size, reduced noise, and superior ability for making complex geometric cuts, the temperature rise in bone due to frictional heating at the blade/tissue interface is still a significant problem. The problem is exacerbated with the use of ultrasonics due to the rapid motion involved as compared to that of traditional reciprocating saws.

U.S. Pat. No. 6,379,371 and U.S. Pat. No. 6,443,969 disclose an ultrasonic bone cutting blade with structure permitting adequate cooling when cutting bone.

### OBJECTS OF THE INVENTION

It is an object of the present invention to provide an ultrasonic bone cutting instrument with improved bone cutting capabilities.

A more particular object of the present invention to provide such an ultrasonic bone cutting instrument that cuts bone more quickly, with greater facility.

A further object of the present invention is to provide such improvements that may also be used for other types of ultrasonic surgical instruments.

These and other objects of the present invention will be apparent from the descriptions and drawings herein. Although every object of the invention is attainable by at least one embodiment of the invention, there is not necessarily any single embodiment that achieves all of the objects of the invention.

### SUMMARY OF THE INVENTION

The present invention recognizes the need which exists for a more efficient ultrasonic bone cutting instrument that expedites bone cutting. The invention is directed in part to an ultrasonic cutting instrument which does not require predrilled holes for cutting, allows complex geometric cuts, has one or more continuous cutting surfaces, and provides for liquid irrigation at the blade/tissue interface. More specifically, the present invention pertains in part to an ultrasonically vibrated cutting instrument with a provision for delivery of a cooling medium for reducing and limiting thermal damage to living tissue. The present invention specifically targets the application of cutting viable bones in surgery, although the device is not exclusive to this application.

The present invention contemplates ultrasonic surgical instruments with multiple tools extending parallel to one another. The tools have respective operating heads and extend parallel to one another, generally side by side. However, one tool could be located concentrically within another tool. In the latter case the tools would have differently configured heads. Where the tools are laterally disposed, the heads might be mirror symmetric.

As discussed in detail hereinafter, an ultrasonic instrument in accordance with the present invention may take the form of a bone cutter, with planar blade bodies disposed adjacent one another, for out-of phase movement in parallel planes.

An ultrasonic surgical instrument in accordance with the present invention comprises at least two tools each including a respective operating head with an operative surface or edge. At least one source of ultrasonic mechanical vibratory energy is provided which produces at least two vibration modes out of phase with one another. The tools are each connected to the source of ultrasonic mechanical vibratory energy for enabling transmission of a respective one of the vibration modes into the respective tool so that the tools are driven out of phase with one another.

The tools typically extend parallel to one another in coextensive relationship. Thus the operative surfaces or edges of the tools are juxtaposed to one another.

The operating heads may be identical to one another. However, in some instruments, the operating heads may be mirror symmetric of concentric. In that latter case, one tool shaft may extend coaxially through the other tool shaft, so that one of the operating heads is surround by the other operating head.

Where the instrument is particularly adapted for bone cutting, the heads of the two tools may take the form of planar cutting blade bodies. Accordingly, an ultrasonic surgical instrument of the bone-cutting variety comprises, in accordance with the present invention, at least two cutting blades each comprising a substantially flat or planar blade body having a cutting edge extending parallel to the cutting edge of the blade body of the other of the at least two cutting blades. The instrument includes at least one source of ultrasonic mechanical vibratory energy having at least two vibration modes out of phase with one another. The at least two cutting blades are each connected to the source of ultrasonic mechanical vibratory energy for enabling transmission of a respective one of the vibration modes into the respective blade body so that the at least two cutting blades are driven out of phase with one another.

The two vibration modes typically have a common ultrasonic frequency. In addition, the two vibration modes are typically 180° out of phase with one another. Alternative phase relationships are possible. For instance, the vibration modes may be out of phase by 90° or a quarter wave. Alternatively, the phase difference may vary as when the activation frequencies differ slightly from one another (e.g., by 1000 Hz).

Pursuant to one embodiment of the present invention, the source of ultrasonic vibratory energy includes a single transducer assembly and a half wave connector horn, one and only one of the shanks of the at least two cutting blades being connected to the transducer assembly via the half wave horn, the other shank being connected directly to the transducer assembly.

Pursuant to another embodiment of the present invention, the source of ultrasonic vibratory energy includes two distinct transducer assemblies each producing one of the vibration modes and connected to a respective one of the at least two cutting blades. The two vibration modes may have one ultrasonic frequency in common and the vibration modes may be 180° out of phase with one another.

The cutting edge of each of the cutting blades preferably includes a smooth continuous cutting edge. The cutting edge is preferably disposed in a single plane and has an arcuate section at the distal end, with a pair of straight sections continuous with the arcuate section at opposite ends thereof. Each shank may be provided with an axially extending bore for the conveyance of cooling fluid to the respective cutting edge, the respective blade body being provided with an axially extending through-slot communicating at one end with the bore.

### BRIEF DESCRIPTION OF THE DRAWINGS

FIG. 1 is an overall view of a surgical system having a dual ultrasonic cutting blade in accordance with the present invention.

FIG. 2 is a schematic partial side elevational view of an ultrasonic cutting instrument having two blades, in accordance with the present invention.

FIG. 3 is a schematic perspective view of one of the blades of FIG. 2

FIG. 4 is partially a block diagram and partially a side elevational view of an alternative embodiment of an ultrasonic cutting instrument having two blades, in accordance with the present invention.

FIG. 5 is a diagram of another dual ultrasonic cutting instrument in accordance with the present invention.

### DETAILED DESCRIPTION

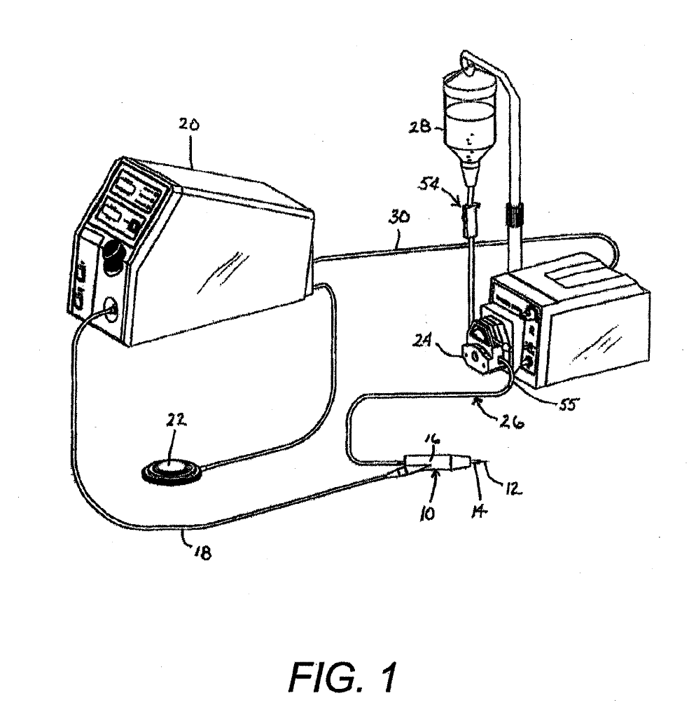

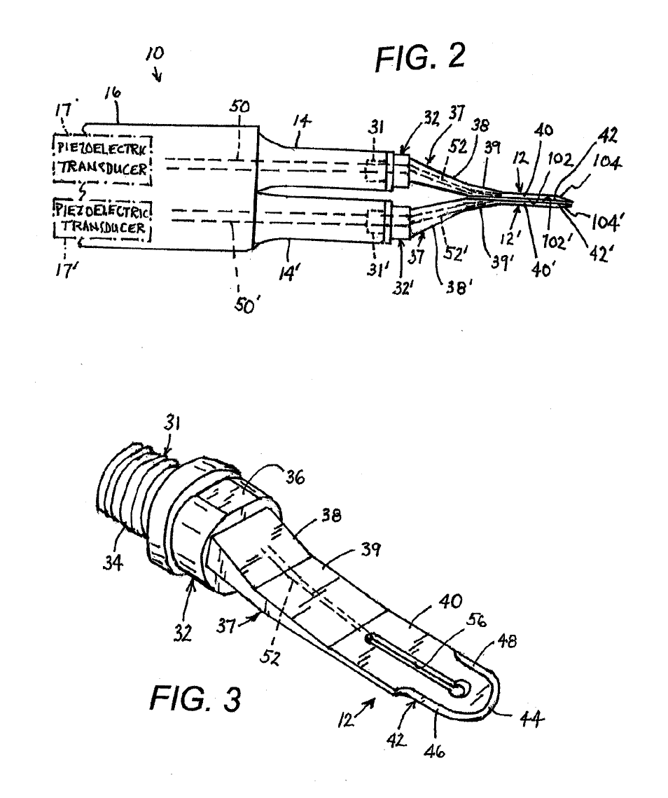

As shown in FIGS. 1 and 2, an ultrasonic surgical system includes a handpiece 10 carrying a pair of laterally juxtaposed tools in the form of bone cutting blades 12 and 12′ disposed in parallel to one another. Handpiece 10 is attached to blades 12 and 12′ via respective probes 14 and 14′ and further includes a housing 16 which encases two piezoelectric crystal assemblies 17 and 17′ each of the kind disclosed in U.S. Pat. No. 5,371,429 to Manna. In response to two sinusoidal oscillating signals transmitted over a cable 18 from an ultrasonic generator 20, the crystal assemblies 17 and 17′ in the handpiece produce longitudinal ultrasonic pressure waves transmitted through probes 14 and 14′ to blades 12 and 12′. Signal generator 20 is activated via a footswitch 22. Handpiece 10 is also connected to an irrigation pump 24 via a tube 26. Pump 24 moves an irrigant liquid from a reservoir or IV type hanging bag 28 through tube 26 to handpiece 10 in response to a signal carried over a cable 30 from signal generator 20 under the control of footswitch 22.

The mechanical vibrations produced by the piezoelectric crystal assemblies 17 and 17′ in handpiece 10 are amplified mechanically via the transducers' shapes and still further by the shapes of probes 14 and 14′ and blades 12 and 12′, using techniques known to those skilled in the art of ultrasonics. Probes 14 and 14′ are attached to handpiece 10 via externally threaded connections 31 and 31′, shown in FIG. 2. Probes 14 and 14 ‘ are thus replaceable by the user to facilitate the use of disposable sterile blades 12 and 12’ from one procedure to the next. Handpiece 10 may be sterilized by autoclaving as well as by other conventional methods. While probes 14 and 14′ can be sterilized, maintaining a good cutting edge and cleanliness is such a key issue that a disposable tip or a disposable tool/tip assembly is envisioned. The primary purpose of probes 14 and 14′ is to mechanically amplify the vibrations from the piezoelectric transducer assemblies 17 and 17′ and transmit those vibrations through to cutting blades 12 and 12.

Blades 12 and 12′ are mirror symmetric and include planar operating heads or blade bodies 40 and 40′ with facing surfaces 102 and 102′ that are planar and closely disposed in parallel with one another. Generally, surfaces 102 and 102′ are spaced from one another by a gap (not designated) that is so narrow as to facilitate the production of a single cut while being conducive to the flow of liquid coolant between blades 12 and 12′.

FIG. 3 depicts cutting blade 12, while cutting blade 12′ is essentially a mirror image thereof. As shown in FIGS. 2 and 3, blades 12, 12′ each include an integral shank portion 32, 32′ having an external screw thread 34 for replaceably mounting the blade to the respective probe 14, 14′. Alternatively, blades 12 and 12′ may be permanently attached to probes 14 and 14′. In the former case, blades 12, 12′ are tightened by a wrench (not shown) applied to wrench flats 36 on shank portions 32, 32′. Blades 12 and 12′ are shaped to amplify the longitudinal vibratory motions. More specifically, blades 12, 12′ include a shaft 37, 37′, intermediate shank 32, 32′ and planar body portion 40, 40′ comprising serial tapered or wedge-shaped sections 38, 38′ and 39, 39′ for focusing or concentrating ultrasonic vibratory energy and transmitting the energy to planar bodies 40, 40′ of blades 12, 12′. Each whole transducer half, horn and tip assembly is designed to resonate in a longitudinal or back and forth type of motion. This motion provides the cutting action at the tips of blades 12, 12′.

Planar blade body portions 40, 40′ are provided at ends opposite tapered portions 38, 38′ and 39, 39′ and shanks 32, 32′ with smooth continuous blade edges 42, 42′ including a central circularly arcuate section 44 (FIG. 3 only) and a pair of linear end sections 46 and 48, all in a single plane for each blade. Blade or cutting edge 44 is sharpened along a full radius of arcuate section 44, as well as along straight sections 46 and 48, with a knife type edge that can smoothly be drawn back and forth in a brushing type motion. This cutting edge structure allows the user to maintain a constant motion at the tip, which has been shown to be important to prevent overheating of the tissue at the surgical site. More particularly, blade edges 42 and 42′ are each beveled along a laterally outward side 104 and 104′ (FIG. 2), facing away from the other blade 12′ and 12, so that the peripheries are closely aligned for facilitating the production of a smooth narrow cut.

As further illustrated in FIGS. 2 and 3, blades 12 and 12′ also incorporate structure providing a path for coolant from irrigation pump 24 (FIG. 1) to reach blade edges 42 and 42′, as well as tissues being cut during a surgical procedure. For conducting irrigant to blade edges 42 and 42′ and the surgical site, probes 14 and 14′ are formed with axial passageways or bores 50 and 50′ which communicate with respective axial passageways or bores 52 and 52′ in blades 12 and 12′ and more particularly in shanks 32 and tapered blade portions 38 and 39. The irrigation fluid is typically a sterile saline solution supplied in hanging bag 28 (FIG. 1). Bag 28 is punctured with a vented IV spike supplied at the end of a sterile tube set 54. The spike allows the fluid to flow into a silicone tube section 55 of tube 26 of set 54. Silicone tube section 55 passes through pump 24 which takes the form of a peristaltic or roller type pump. Pump 24 pushes the fluid along tube 26 to a connection at the handpiece 10. The fluid travels through an integral channel inside the handpiece 10, as described in U.S. Pat. No. 5,371,429. From handpiece 10 the fluid travels through probes 14 and 14′ to blades 12 and 12′.

The disclosures of U.S. Pat. Nos. 6,379,371 and 6,443,969 are hereby incorporated by reference to explicate further the possible structures of blades 12 and 12′. For instance, each blade 12, 12′ may include a longitudinal or axially extending through-slot 56 in the respective planar body portion 40, 40′ facilitating fluid distribution not only along cutting edges 42, 42′ but also throughout the gap between blade bodies 40, 40′ (and tapered blade sections 39, 39′).

Probes 14 and 14′ extend side by side and parallel to one another. Probes 14 and 14′ have respective shafts, heads (blades 12 and 12′) and coupling shanks 32.

Tools or blades 12 and 12′ and more particularly planar blade body portions 40 and 40′ thereof extend parallel to one another in coextensive relationship. Thus the operative surfaces or edges 42 and 42′ of the tools are juxtaposed to one another. Planar blade bodies 40 and 40′ are disposed adjacent one another, for out-of phase movement in parallel planes. Piezoelectric crystal assemblies 17 and 17′ are transducers that function as respective sources of ultrasonic mechanical vibratory energy producing respective vibration modes that are out of phase with one another. Ultrasonic signal generator 20 produces dual out-of-phase electrical waveforms that are separately fed to transducers or crystal assemblies 17, 17′. Shanks 31 and 31′ of tools or blades 12 and 12′ are respectively connected to piezoelectric crystal assemblies 17 and 17′ for enabling transmission the respective modes of mechanical vibratory energy into the respective tool shafts so that the tools or blades 12 and 12′ are driven out of phase with one another.

The two vibration modes of crystal assembly transducers 17, 17′ typically have a common ultrasonic frequency. In addition, the two vibration modes are typically 180° out of phase with one another. Alternative phase relationships are possible. For instance, the vibration modes may be out of phase by 90° or a quarter wave. Alternatively, the phase difference may vary continuously by having the activation frequencies differ slightly from one another (e.g., by 1000 Hz). The frequency difference is enough to enable phase shifting but not enough to appreciably affect the resonance or standing wave characteristics of the blades 12, 12′.

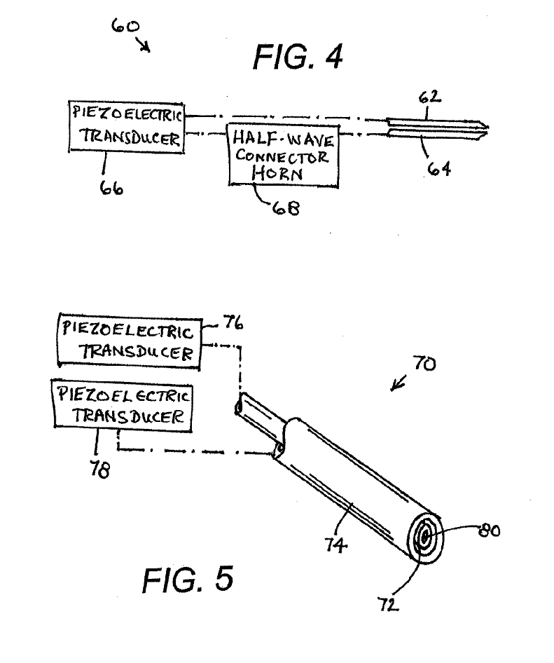

As depicted in FIG. 4, an ultrasonic surgical instrument 60 includes two cutting blades 62, 64 and a singular source 66 of ultrasonic vibratory energy in the form of a piezoelectric transducer assembly. One cutting blade 62 is connected directly to transducer assembly 66. The other cutting blade 64 is connected indirectly to transducer assembly 66 via a half wave connector horn 68. Half-wave horn 68 causes blade 64 to oscillate 180° out of phase with blade 62.

FIG. 5 schematically depicts an ultrasonic surgical instrument 70 including two concentrically or co-axially disposed tools or probes 72 and 74 each operatively connected to a respective source 76, 78 of ultrasonic waveform energy such as a piezoelectric crystal array. Sources 76 and 78 produce ultrasonic standing waves in tools or probes 72, 74 that are out of phase with one another. Surgical instrument may serve as a bone drill, with a central suction channel 80.

Although the invention has been described in terms of particular embodiments and applications, one of ordinary skill in the art, in light of this teaching, can generate additional embodiments and modifications without departing from the spirit of or exceeding the scope of the claimed invention. For instance, it is possible for an ultrasonic instrument assembly to incorporate plural ultrasonic instrument tools with respective vibration modes that are out of phase with one another by a quarter wave (90°) or some other magnitude. Moreover, the phase shift might be varying, for instance, where the vibration modes are of different frequencies. Accordingly, it is to be understood that the drawings and descriptions herein are proffered by way of example to facilitate comprehension of the invention and should not be construed to limit the scope thereof.

What is claimed is:

1. An ultrasonic surgical instrument comprising: at least two cutting blades each comprising a substantially flat or planar blade body, each said blade body having a cutting edge extending parallel to the cutting edge of the blade body of the other of said at least two cutting blades; and at least one source of ultrasonic mechanical vibratory energy having at least two vibration modes out of phase with one another, said at least two cutting blades each being operatively connected to said at least one source of ultrasonic mechanical vibratory energy for enabling transmission of a respective one of said vibration modes into the respective blade body so that said at least two cutting blades are driven out of phase with one another.

2. The surgical instrument defined in claim 1 wherein said at least two vibration modes have a common ultrasonic frequency.

3. The surgical instrument defined in claim 2 wherein said at least two vibration modes are 180° out of phase with one another.

4. The surgical instrument defined in claim 3 wherein said one source includes a single transducer assembly and a half wave connector horn, one and only one of the shanks of said at least two cutting blades being connected to said transducer assembly via said half _{wave} horn.

5. The surgical instrument defined in claim 1 wherein said at least one source of ultrasonic mechanical vibratory energy including two distinct transducer assemblies each producing one of said vibration modes and connected to a respective one of said at least two cutting blades.

6. The surgical instrument defined in claim 5 wherein said at least two vibration modes have a common ultrasonic frequency.

7. The surgical instrument defined in claim 6 wherein said at least two vibration modes are 180° out of phase with one another.

8. The surgical instrument defined in claim 1 wherein the cutting edge of each of said at least two cutting blades is a smooth continuous cutting edge.

9. The surgical instrument defined in claim 8 wherein said cutting edge is disposed in a single plane and has an arcuate section.

10. The surgical instrument defined in claim 9 wherein said cutting edge includes a pair of straight sections continuous with said arcuate section at opposite ends thereof.

11. The surgical instrument defined in claim 1 wherein each said shank is provided with an axially extending bore for the conveyance of cooling fluid to the respective cutting edge, the respective blade body being provided with an axially extending through-slot communicating at one end with said bore.

12. An ultrasonic surgical instrument comprising: at least two tools each comprising a respective operating head having an operative surface or edge; and at least one source of ultrasonic mechanical vibratory energy having at least two vibration modes out of phase with one another, said at least two tools each being operatively connected to said at least one source of ultrasonic mechanical vibratory energy for enabling transmission of a respective one of said vibration modes into the respective tool so that said at least two tools are driven out of phase with one another.

13. The surgical instrument defined in claim 12 wherein said at least two tools extend parallel to one another in coextensive relationship.

14. The surgical instrument defined in claim 13 wherein the operating heads of said at least two tools are identical to one another.

15. The surgical instrument defined in claim 14 wherein the operating heads of said at least two tools are planar cutting blade bodies.

16. The surgical instrument defined in claim 12 wherein vibration modes have a common ultrasonic frequency.

17. The surgical instrument defined in claim 16 wherein said at least two vibration modes are 180° out of phase with one another.

18. The surgical instrument defined in claim 12 wherein said one source includes a single transducer assembly and a half wave connector horn, one and only one of said at least two cutting blades being connected to said transducer assembly via said half wave horn.

19. The surgical instrument defined in claim 12 wherein said at least one source of ultrasonic mechanical vibratory energy includes two distinct transducer assemblies each producing one of said vibration modes and connected to a respective one of said at least two tools.

20. The surgical instrument defined in claim 19 wherein said at least two vibration modes have a common ultrasonic frequency.

21. The surgical instrument defined in claim 20 wherein said at least two vibration modes are 180° out of phase with one another.
